(19) United States
(12) Patent Application Publication
(10) Pub. No.: US 2003/0171680 A1
(43) Pub. Date: Sep. 11, 2003

(54) Needle and sensor adapters for medical systems

(76) Inventor: Yoav Paltieli, Haifa (IL)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/404,050

(22) Filed: Apr. 2, 2003

Related U.S. Application Data
(63) Continuation of application No. 09/830,295, filed on Jul. 23, 2001, now abandoned.
Application No. 09/830,295 is a 371 of international application No. PCT/IL99/00560, filed on Oct. 24, 1999.

(30) Foreign Application Priority Data
Oct. 26, 1998 (IL) 126742

(51) Int. Cl. A61B 8/14

(52) U.S. Cl. 600/459

(57) ABSTRACT

A needle adapter is provided having a first member including a head portion and a tail portion, the head portion having a bore extending at least substantially therethrough. The head portion is configured for receiving a segment of a second member and is also configured for receiving a needle. The second member has a segment and a tail portion, the segment attached to the tail portion and adapted for receipt in the head portion of the first member, for attaching the first member and the second member in a hinge-like manner. This attachment allows for movement of the first and second members between open and closed positions. The tail portions of the first member and the second member are configured such that when the closed position is achieved, a volume for retaining a sensor is formed. There is also a pin, removably attachable from the bore of the head portion of said first member. This pin is for securing the needle upon its mounting in the needle adapter.

### CROSS-REFERENCE TO RELATED APPLICATIONS

This application is a continuation application of U.S. patent application Ser. No. 09/830,295, filed Jul. 23, 2001, which claims priority from PCT Patent Application PCT/IL99/00560, filed Oct. 24, 1999, published as WO 00/24306 on May 4, 2000 in the English language under PCT Article 21(2), which in turn claims priority from Israel Patent Application 126742, filed Oct. 26, 1999, all three of which are hereby incorporated by reference.

### FIELD OF THE INVENTION

The present invention is directed to guidance systems associated with imaging devices, such as ultrasound, CT, etc., and in particular to needle holders and adapters thereon for attaching sensors thereto and adapters for attaching sensors to the imaging device, such as an ultrasound transducer, CT imager, etc.

### BACKGROUND OF THE INVENTION

Imaging methods, such as ultrasound, CT, MRI, x-ray, etc., are used to guide the insertion of surgical tools for diagnosis and therapy. For example, ultrasound imaging and procedures associated therewith are routinely performed in association with fetal monitoring and development, breast biopsy, other biopsies, aspirations and other interventional insertions.

Systems for performing Interventional procedures with imaging devices are disclosed in the assignee's U.S. Pat. No. 5,647,373 and PCT International Application, Publication No. WO 97/03609 (International Publication Number PCT/IL96/00050), entitled: FREE-HAND AIMING OF A NEEDLE GUIDE, both of these documents being incorporated by reference in their entirety herein. In the systems disclosed therein, an imaging device, such as an ultrasound transducer functions in cooperation with a guidance system to monitor the relative position of a needle (or any other similar invasive device) with respect to the imaging device. This produces an image on a screen or monitor, showing the internal surgical site and the needle position within the body relative to the imaging device. PCT WO 97/03609 also discloses sensors on the needle and/or the imaging device, here an ultrasound transducer, to further assist in guiding the needle on the screen or monitor.

### SUMMARY OF THE INVENTION

The present invention improves the contemporary art medical interventional procedures by providing needle-holding devices including adapters for attaching sensors thereto and adapters for imaging devices, such as ultrasound transducers, for attaching sensors thereto.

The needle adapters and needle guide are suitable for use with needles of various gauges. The term “needle” is used throughout this application, to describe needles, that include, for example, standard medical needles, biopsy needles, ablation devices (such as those in cryo devices), biopsy guns (such as MAMMOTOME® type biopsy guns-available as part of MAMMOTOME® Vacuum Biopsy System available from Biopsys Ethicon Endo-Surgery), introducers, or other similar medical interventional devices.

The design of the needle adapters and guide minimize the chance of incorrect insertion of the needle and the position sensors in the adapters whereby the chances of incorrectly inserting the sensor(s) is extremely difficult.

The needle adapters and guide provide housings for a sensor or sensors, that substantially restrain sensor movement, essentially negating any movement.

The construction of the needle adapters and needle guide enable fast and easy release of the sensors from the adapters after opening the adapters.

The design of the adapters enables the construction of single use adapters or multiple use adapters upon choice.

The design and structure of the first needle adapter and of the needle guide enables correct knowledge of the position of the needle and needle tip with respect to the position sensor, without the need to calibrate the position sensor to the needle. The design and structure of the second needle adapter enables correct knowledge of the orientation and trajectory of the needle with respect to the position sensor without the need for calibrating the position sensor to the needle.

The first needle adapter and the needle guide ensures a firm lock on the needle after closing it, therefore ensuring constant needle positioning with respect to the sensor during the procedure.

The first needle adapter enables attaching the needle to the needle adapter without the risk of contacting foreign particles on the needle tip or on other parts of the needle which enter the body.

The second needle adapter, and especially the needle guide, minimize the bending of the needle during an invasive procedure.

The device adapter enables attaching position sensors to any type of ultra-sound transducers or other imaging devices.

The device adapter enables firm and constant positioning of the sensor with respect to larger invasive devices such as biopsy guns.

The construction of the device adapter ensures that once the sensor is calibrated to the transducer, calibration parameters (as described in the PCT No. WO 97/03609) are saved, the position of the sensor with respect to the ultra-sound transducer (or other imaging device) for each subsequent procedure is correctly known, without the need of additional calibrations (until the adapter is moved/removed). This also allows for multiple insertion and removal of the sensors in the adapters.

The present invention provides a needle adapter having a first member including a head portion and a tail portion, the head portion having a bore extending at least substantially therethrough. The head portion is configured for receiving a segment of a second member and is also configured for receiving a needle. The second member has a segment and a tail portion, the segment attached to the tail portion and adapted for receipt in the head portion of the first member, for attaching the first member and the second member in a hinge-like manner. This attachment allows for movement of the first and second members between open and closed positions. The tail portions of the first member and the second member are configured such that when the closed position is achieved, a volume for retaining a sensor is formed. There is also a pin, removably attachable from the bore of the head portion of said first member. This pin is for securing the needle upon its mounting in the needle adapter.

The invention also provides a needle adapter having a first portion, for holding a needle, in communication with a second portion, for housing a sensor. The second portion includes a first member having a head portion and a tail portion, with the head portion configured for receiving a segment of a second member. The second member includes a segment and a tail portion, the segment is adapted for receipt by the head portion of the first member, such that the first member attaches to the second member in a hinge-like manner, allowing for movement of the first and second members between open and closed positions. The tail portions of both the first member and the second member are configured such that when the closed position is achieved, a volume for retaining a sensor is formed.

The invention also provides a system for mounting a sensor including a base member having upper and lower surfaces, a platform over the upper surface of the base member, this platform including oppositely disposed rails. There is also included wings for attaching to oppositely disposed sides of a sensor housing, the wings being configured to be received and retained by the rails. There is a hook intermediate the rails on the platform for retaining the sensor housing thereon, when the wings are retained in the rails.

The present invention additionally provides a needle guide having needle locker and a needle holder below the needle locker and in alignment with the needle locker. There is also a sensor housing in communication with the needle locker.

### BRIEF DESCRIPTION OF THE DRAWINGS

The present invention will be described with respect to the accompanying drawings, where like reference numerals identify corresponding or like components. In the drawings:

FIG. 1 shows the present invention in use in a medical procedure;

FIG. 2A is a partially exploded view of a first embodiment of a needle adapter of the present invention with its component parts separated from each other;

FIG. 2B is a perspective rear view of the first embodiment of the needle adapter in an open position;

FIGS. 3A and 3B detail embodiments for the teeth of the flanges used in locking the adapter of FIGS. 2A and 2B;

FIG. 4 is the perspective view of the apparatus of FIG. 2B, with a sensor loaded into the needle adapter in the open position;

FIG. 5A is a perspective front view of the first embodiment during needle loading;

FIG. 5B is a multi-level top. cross-sectional view of the first embodiment during needle loading;

FIGS. 6A and 6B are perspective views of the needle adapter of the present invention in the closed position, accommodating a sensor and a needle;

FIG. 6C is a cross-sectional view taken along line 6C-6C of FIG. 6B;

FIG. 7A is a perspective view of a device adapter of the present invention;

FIG. 7B is a perspective of the sensor and the housing suited as used with the device adapter of the present invention;

FIG. 8A is a front view of the sensor device as mounted on an ultrasound transducer;

FIG. 8B is a perspective view of a biopsy gun with the sensor device of the present invention mounted thereon;

FIG. 9 is a perspective view of a second embodiment of a needle adapter of the present invention with its component parts separated from each other;

FIG. 10 is a perspective view of the second embodiment of a needle adapter of the present invention;

FIG. 11 is a perspective view of the needle housing of the needle adapter of FIGS. 9 and 10;

FIG. 12A shows the needle adapter of FIGS. 9 and 10 in an exemplary operation;

FIG. 12B shows an alternate embodiment of the needle adapter of FIGS. 9 and 10 in an exemplary operation;

FIG. 12C is a perspective view of a biopsy gun with the sensor adapter of the second embodiment of the present invention mounted thereon;

FIG. 13 is a perspective view of a third embodiment of a needle adapter of the present invention; and

FIG. 14 is a perspective view of the needle trajectory positioner of the third embodiment of the needle adapter of FIG. 13; and

FIG. 15 shows the needle adapter of FIG. 13 in an exemplary operation;

FIGS. 16A and 16B are perspective and side views of an embodiment of the needle adapter of the present invention adapted for receiving a MAMMOTOME® biopsy “gun”;

FIG. 17A is a perspective view of an embodiment of the needle adapter of the present invention operating with a Senorex™ introducer; and

FIG. 17B is a perspective view of the embodiment of the needle adapter of FIG. 17A.

### DETAILED DESCRIPTION OF THE DRAWINGS

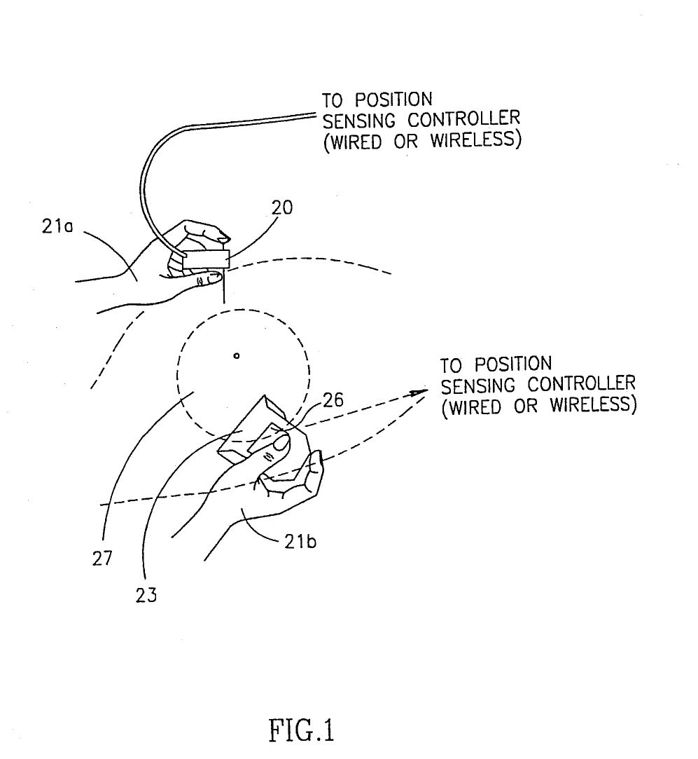

FIG. 1 shows the apparatus of the present invention in use with an interventional imaging system as described in PCT Application No. WO 97/03609 (PCT/IL/96/00050). The interventional imaging system shown uses an ultrasound transducer for generating the requisite image. This interventional imaging system is exemplary only, for the apparatus of the present invention can be used with other interventional imaging systems, besides those disclosed in PCT Application No. WO 97/03609.

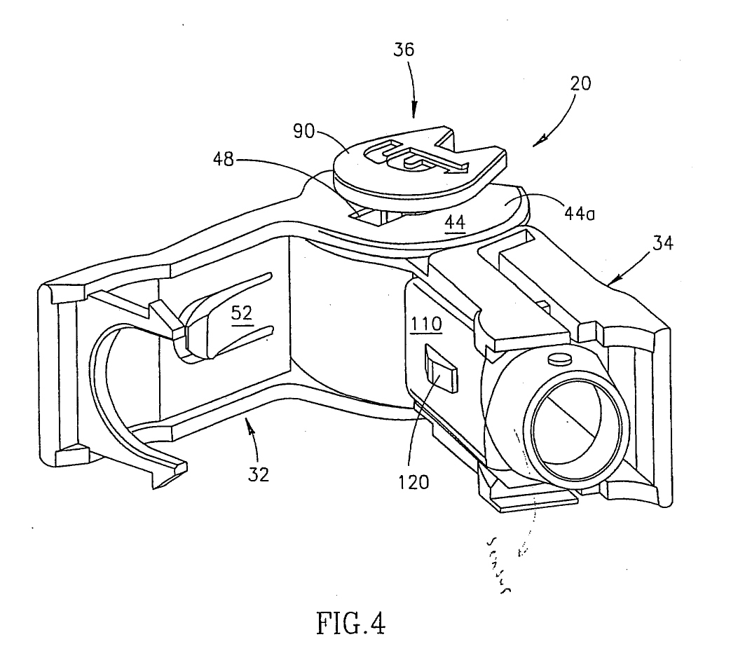

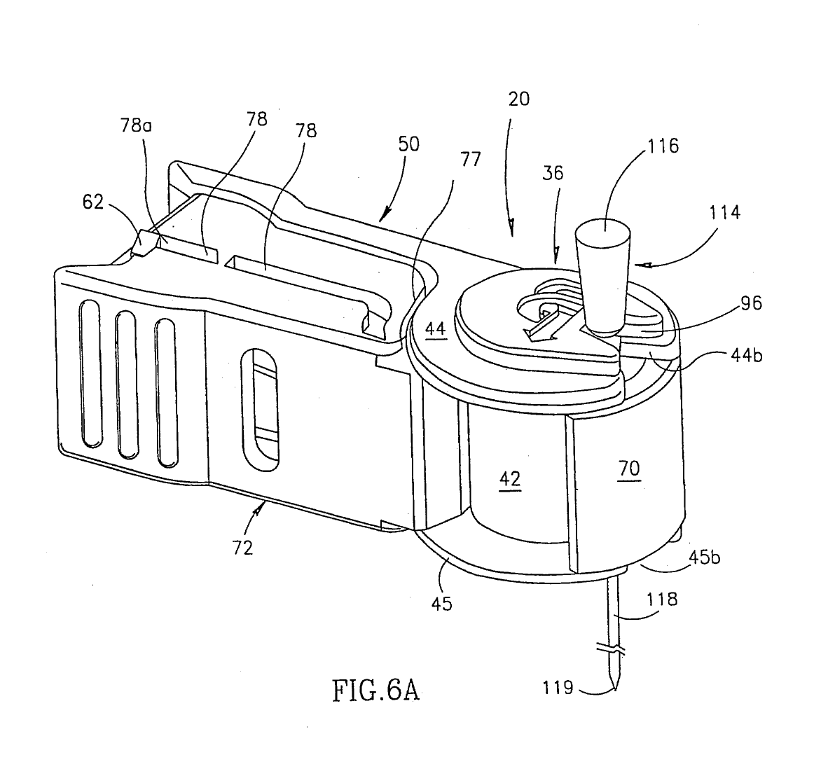

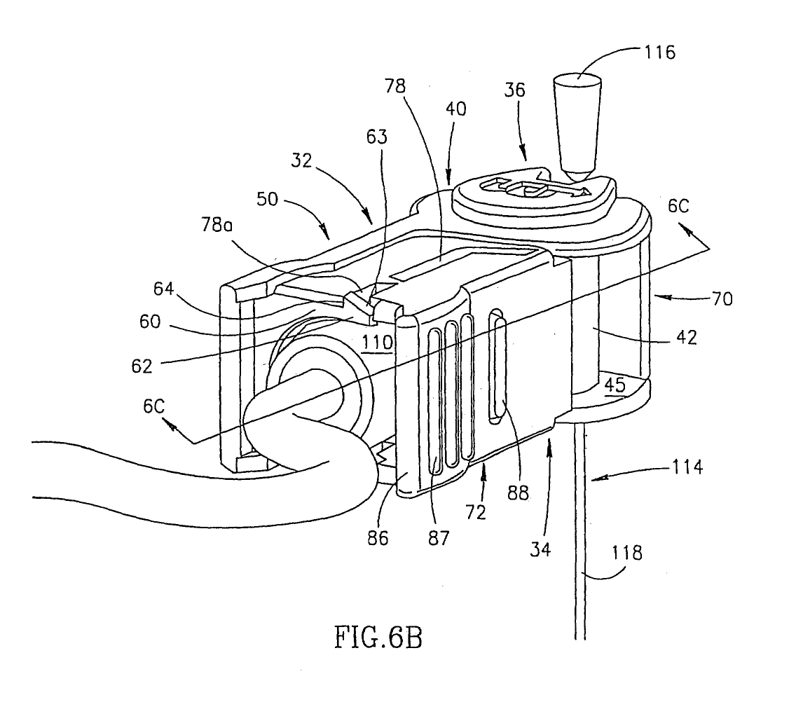

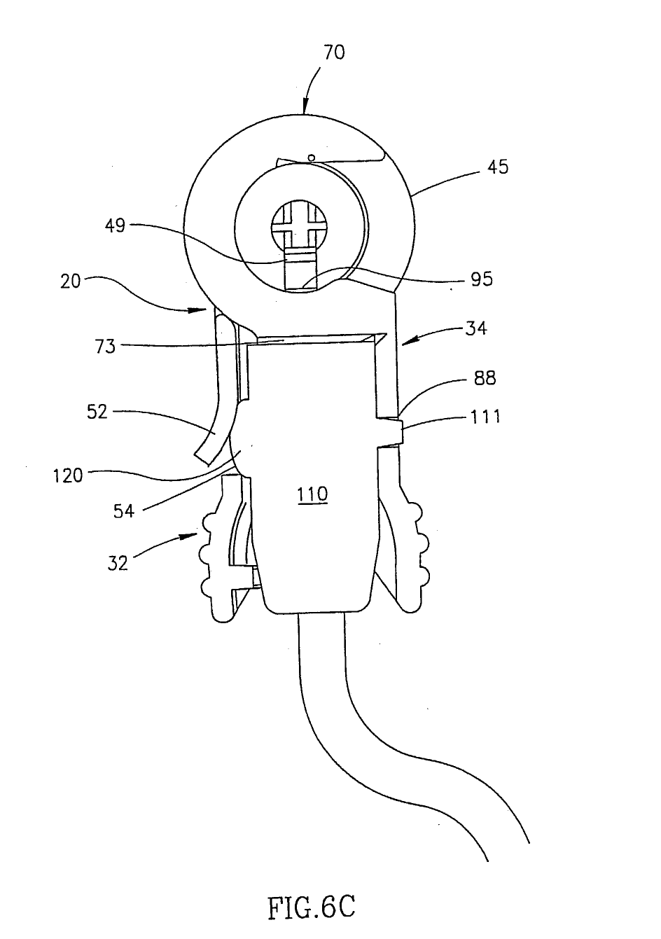

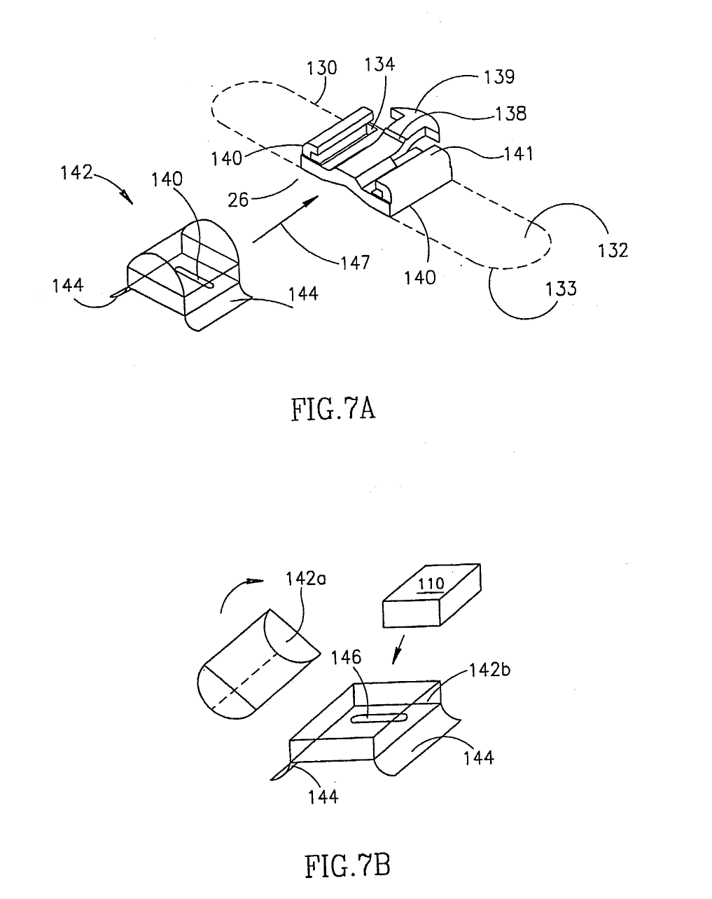

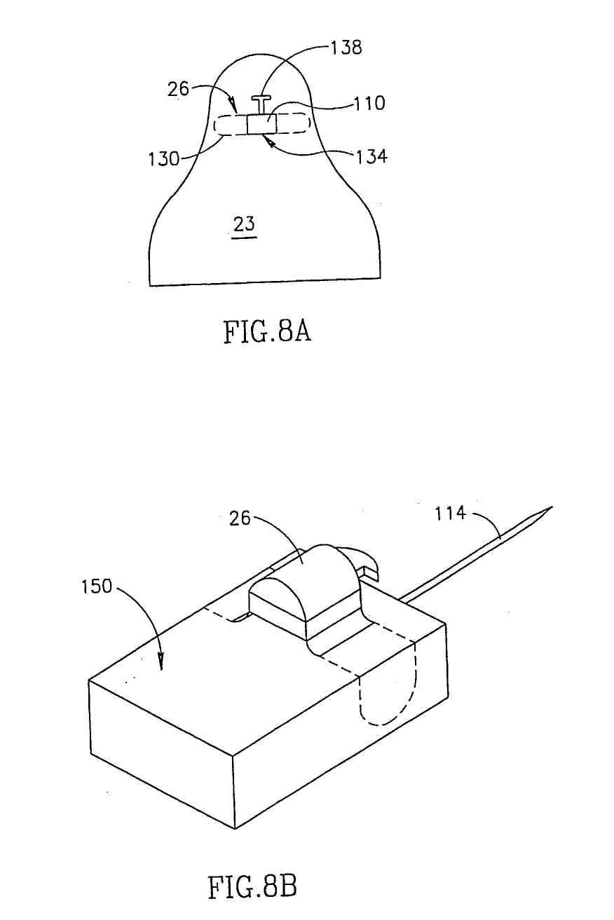

Apparatus of the present invention is shown in operation, as a surgeon holds the needle adapter 20, having a sensor 110 (FIGS. 4, 6B and 6C), typically a position sensor as described in PCT Application No. WO 97/03609, that can be linked to the position sensing controller by wired or wireless communication, in accordance with PCT Application No. WO 97/03609. This adapter 20 is accommodated in a first hand 21a of a surgeon. An ultrasound transducer 23 having a device adapter 26 for accommodating a sensor 110 (FIGS. 7 and 8), typically a position sensor as described in PCT Application No. WO 97/03609, that can be linked to the position sensing controller by wired or wireless communication, in accordance with PCT Application No. WO 97/03609. The ultrasound transducer 23 is in the second hand 21b, as the surgeon is operating on a surgical site 27, for example, a female breast biopsy.

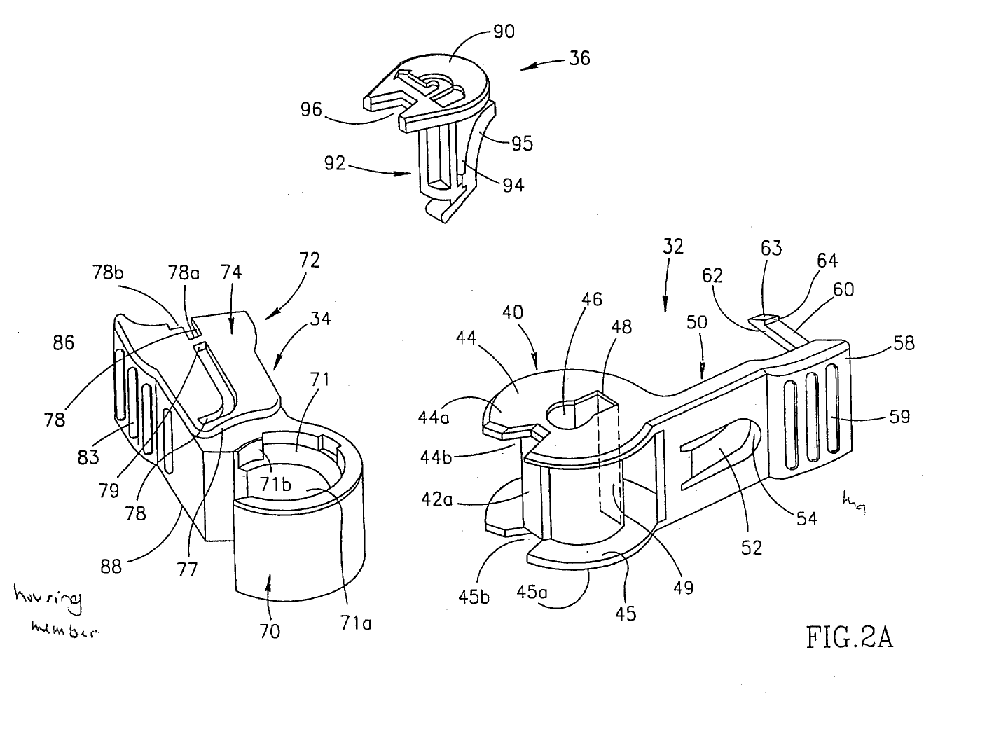

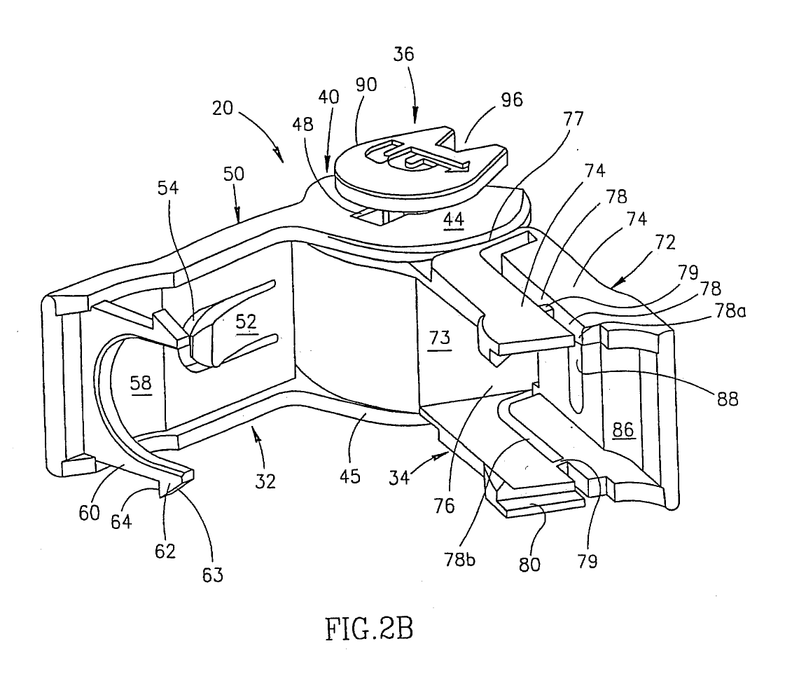

FIG. 2A shows the needle adapter 20, divided into three pieces, a main body 32, a housing member 34 and a pin 36. These pieces 32, 34, 36 are configured, such that when assembled, provide a self-securing (and locking) structure absent any additional adhesives or fasteners, although additional adhesives or fasteners are also permissible if desired. While three pieces are preferred, any number of other pieces are also suitable. It is preferred that these pieces 32, 34, 36 be made of plastic, elastomers or the like, by techniques such as injection molding or the like.

Turning also to FIG. 2B, the main body 32 has a head 40, formed by a cylinder 42 intermediate platforms 44, 45. The cylinder 42 includes a bore 46, that extends from an opening 48 in the platform 44. The bore 46 preferably extends substantially through the cylinder 42 to the platform 45. However, this platform 45 may have an opening, such that the head 40 would have an opening (bore) extending therethrough.

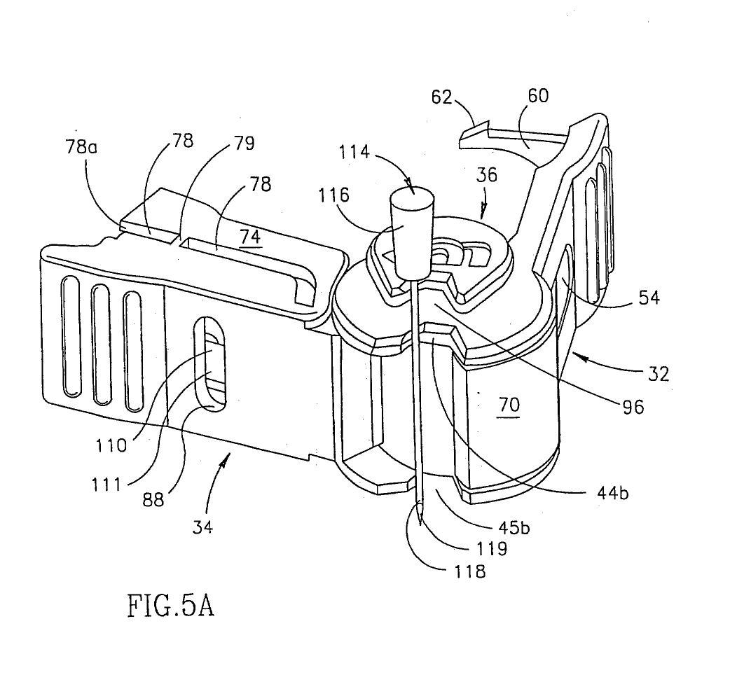

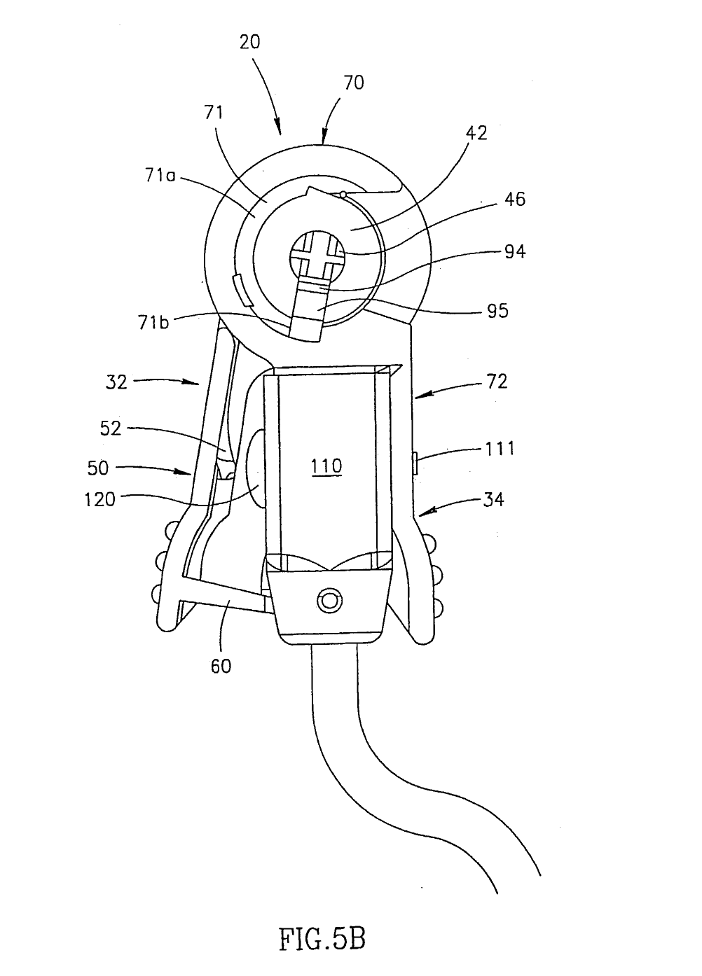

The opening 48 and bore 46 are preferably coaxial and of the same shape, such as key-hole, rounded or the like, to accommodate the pin 36. The platforms 44, 45 extend beyond the cylinder 42, these portions of the platforms extending beyond the cylinder 42 for supporting the claw 70 of the housing member 34. The platforms 44, 45 have outer surfaces 44a, 45a and preferably include cut outs 44b, 45b, that combine with a preferably flattened wall 42a of the cylinder to accommodate the needle 114 (FIGS. 5A, 6A and 6B). The cylinder 42 also includes a slot 49 (shown in broken lines) that preferably extends the length of the bore (although other lengths are also suitable) to accommodate the arm 94 of the pin 36, this arm 94 designed to extend through this slot 49 when the pin 36 is placed into the bore 46 (as shown in FIG. 5B).

A tail 50 extends from the head 40. A tongue 52 is preferably formed in this tail 50, that is resilient and behaves in a spring-like manner (due to the nature of the material from which the main body 32 is made). It is movable into an opening 54, when a sensor 110 is placed into the assembled adapter 20 (as shown in FIGS. 4-6B). The tail 50 preferably includes an outwardly extending end 58, that may include ribs 59 thereon, for providing gripping ease to the surgeon. Flanges 60 extend from the tail 50, preferably from the outwardly extending end 58, having teeth 62 configured for engaging edges 78a (see also FIG. 6b) of the walls 74 of the housing member 34, to hold the adapter 20 in place in a secure manner such that the sensor 110 (FIG. 6B) can be properly retained therein.

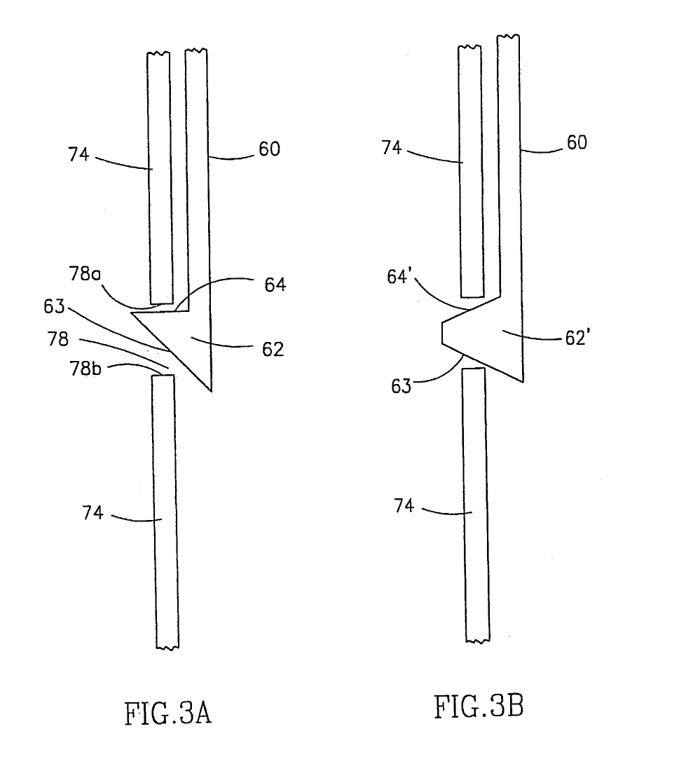

As shown in FIG. 3A, the teeth 62 include a tapered portion 63 and a substantially flat platform 64, so as to be one-way biased. This one-way biasing allows for the flange 60 supporting the teeth 62 to flex inward against the troughs 80 in the walls 74, and snapping into place in the tracks 78 when the platforms 64 pass the upper edges 78a of the walls 74, when the adapter is assembled into a closed position (by moving the respective tails 50, 72 toward each other as claw 70 of the housing member 34 pivots about the cylinder 42 of the main body 32). This tooth design (of FIG. 3A) limits the needle adapter 20 to a single or one-time use, as upon disassembly of the adapter 20 (to the open position), by separating the main body 32 from the housing member 34, by moving their respective tails 50, 72 apart, a portion of the housing member 34 breaks, as the teeth 62 pull a portion of the housing member 34. Should a reusable apparatus be desired, the teeth 62′ may have tapered platforms 64′, as shown in FIG. 3B, or these platform and taper(s) may be rounded.

The housing member 34 includes a claw 70 attached to a tail 72. The claw 70 is adapted to wrap around a substantial portion of the cylinder 42, to contain the needle 114 in the cut outs 44b, 45b of the platforms 44, 45 (as shown in FIG. 6A). This wrapping forms a hinge-like attachment for the main body 32 and the housing member 34. Preferably, the claw 70 is of a radius of curvature, equal to or just slightly smaller than the radius of curvature of the cylinder 42, such that the claw 70 attaches to the cylinder 42 by a frictional engagement, allowing for the main body member 32 and the housing member 34 to be moveable relative to each other about the cylinder 42. The claw 70 is of sufficient flexibility to accommodate several type of gauge needles. For example, the adapter 20 can be made to accommodate needle gauges typically of 14-18 and 18-23.

The claw 70 also includes a shoulder 71, indented therein, running along substantially all of the perimeter at an upper portion thereof, along the inner wall 71a. This shoulder 71 terminates at a stop surface 71b, that extends inward and beyond the shoulder 71.

The tail 72 includes a stop wall 73, proximate the claw 70, and laterally disposed walls 74, the walls 73, 74 defining a cavity 76. This cavity 76, is of a volume sufficient to accommodate a sensor 110 (FIGS. 4-6C). The walls 74 include curved segments 77, proximate the head 72, to accommodate the curvature of the platforms 44, 45 and thus, accommodate the pivotal movement of the housing member 34 relative to the main body 32.

Tracks 78 preferably are cut into the walls 74 dividing them into two parts. The tracks 78 define edges 78a, 78b of the walls 74. The tracks 78 preferably run a substantial length along the walls 74 (in a direction from claw 70 to tail 72) and a bridge 79 connects the parts of the walls 74. The tracks 78 coupled with the bridge 79 serves to facilitate breakage of this housing member 34, should a single use adapter be desired (as detailed above). However, the track lengths may be altered as desired, should a multiple use adapter be desired, with tracks 78 only necessary at the end of the tail 72, to accommodate the teeth 62 for locking the adapter 20, as detailed below.

Troughs 80 at the end portion of the tail 72 are cut into the walls 74, and are position such that the teeth 62 ride in them when the adapter 20 is moved to the closed position. The closed position is reached when the teeth 62 pass beyond the edge 78a and flex outward. The platforms 64 of the teeth 62 ultimately engage the respective edges 78a, to form a snap fit between the main body 32 and housing member 34.

Similar to the tail 50, of the main body 32, the tail 72 preferably includes an outwardly extending end 86, that may include ribs 87 thereon, for providing gripping ease to the surgeon. The tail 72 also includes an opening 88, extending therethrough, for accommodating a bottom fin 111 of a sensor 110 (as shown in FIG. 6C), in order to properly place the sensor 110 in the adapter 20.

The pin 36 includes a head 90 attached to a shaft 92, the shaft including an arm 94. The head 90 typically includes a cut-out 96, preferably corresponding in shape to the cut outs 44b, 45b of the platforms 44, 45.

The shaft 92 and arm 94 are configured to fit through the opening 48 in the first platform 44 and the bore 46 in the cylinder 42. The arm 94 is formed on the end of the shaft 92 in a manner whereby it is preferably integral therewith and pivotal thereon in a spring-like manner thereon. This arm 94 includes an outer extension 95 that extends in the direction of the head 90. The extension 95 is of a width that enables it to extend through the slot 49 after the pin 90 has been inserted into the bore 46. This extension 95 locks the pin 36 inside the cylinder 42 by abutting the platform 44.

Continuing with FIGS. 2A and 2B and turning now to FIGS. 4, 5A and 5B, an example operation of the needle adapter 20 is shown. As shown in FIGS. 2B and 4, the claw 70 (FIG. 2A) of the housing member 34 is placed into engagement with the cylinder 42 of the main body 32. The now formed apparatus 20 is in an open state, with the tails 50, 72 of the respective main body 32 and housing member 34 spread apart. The pin 36 is inserted into the platform opening 48 and into the bore 46, such that its head 90 rests above the outer surface 44a of the platform 44. The extension 95 of the arm 94 now rests within or on the shoulder 71 of the claw 70, and extends through the slot 49 in the cylinder 42.

A sensor 110 (detailed above), is now placed into the housing member 34 in the cavity 76, such that its bottom fin 111 fits into opening 88 (shown in FIG. 6C) and its rear side rests proximate or against to the stop wall 73. The cavity 76 is preferably of dimensions just slightly larger than the sensor 110, to accommodate it in a snug, preferably frictionally snug, manner.

In FIG. 5A, a needle 114, having a head 116, shaft 118 and tip 119, is placed with its shaft 118 in the cut-outs 96, 44b, 45b of the pin 36, and platforms 44, 45, respectively. The needle 114 can be positioned from the side of the adapter 20, avoiding the risk of contacting foreign particles on the needle tip 119, when the needle is inserted into the adapter 20.

Turning to FIG. 5B, the tails 50, 72 of the main body 32 and housing member 34 are brought together, into a semi-closed position, by rotating the claw 70 of the housing member 34 about the cylinder 42 of the main body 32. This rotation continues until the extension 95 of the arm 94, contacts the stop surface 71b of the claw 70. In moving to this semi-closed state, the claw 70 moves over the needle shaft 118 surrounding it.

In order to achieve the closed position and lock the needle 114 and the sensor 110, as shown in FIGS. 6A-6C, it is necessary to push the pin 36 further into the bore 46, by pushing the needle head 116, and to rotate the claw 70 about the cylinder 42. Once the pin 36 has been pushed further into the bore 46, to a point where the extension 95 disengages the stop surface 71b, further rotation of the claw 70 about the cylinder 42 can occur, such that the adapter 20 is moved into the closed position. In moving to the closed position, the teeth 62 ride in the respective troughs 80 and flex outward upon passing into the tracks 78, flex outward such that the platforms 64 extend beyond the edges 78a of the walls 74, locking the adapter 20. At this position, the arm 94 of the pin 36 is held firmly against the inner wall 71a of the claw 70, locking the pin 36. The adapter 20 is now fully assembled (housing the sensor 110) and the needle 114 is in the operative position.

As a result of this locking arrangement, the needle 114 is locked between the claw 70 and the cylinder 42 (flat portion 42a) with its head 116 resting on the head 90 of the pin 36. On each needle insertion, the needle 114 will be at the same position relative to the sensor 110, eliminating the need for calibrating the sensor to the needle 114, prior to performing an invasive procedure. Also, when the adapter 20 is locked, the upper fin 120 of the sensor 110 pushes the tongue 52, such that the tongue 52 and fin 120 fit in the opening 54.

In order to open the adapter 20 (move it to the open position), and release the sensor 110, the tails 50, 72, are spread apart from each other. In the case of a single or one time use adapter, the teeth 62 pull the parts of the walls 74, whereby at least a portion of the housing member 34 breaks. Simultaneously, the tongue 52 springs inward, pressuring the upper fin 120 of the sensor 110, and pushing the sensor 110 from the adapter 20. This ensures rapid and easy release of the sensor 110 from the adapter 20 after opening the adapter 20.

In the case of a multiple use adapter, spreading the tails 50, 72 apart causes the teeth 62′ (shown in FIG. 3B) to slip along the troughs 80. This keeps the adapter 20 intact.

In an alternate embodiment of the above described needle adapter, the main body 32 and housing member 34 would remain in accordance with that detailed above. The pin 36 would be omitted, such that the adapter 20 would be locked with a single movement (pushing the main body 32 and housing member 34 into engagement such that the teeth 62 move into the respective tracks 78 with their platforms 64 in abutment with the upper edges 78a of the walls). With the needle 114 in the cut outs 44b, 45b, the needle 114 would then be pushed downward (toward the apparatus 20) until the head 116 rests on the platform 44 of the apparatus 20.

The device adapter 26 is detailed in FIGS. 7A and 7B. This adapter 26 includes a base 130 having upper 132 and lower 133 surfaces. The upper surface 132 accommodates a platform 134 for the sensor 110, while the lower surface 133 attaches to the device, typically by adhesive attached to portions of this lower surface 133. The adhesive portions may be covered by a cover (not shown) of a material inert to the adhesive, so as not to expose and potentially contaminate the adhesive until use. Alternately, other equivalent fastening arrangements are also permissible. The size of the base 130 may be varied depending upon the transducer, imaging or scanning device desired.

A safety hook 138 that is made of a resilient material, such as plastic, and having a tail 139, is attached to the platform 134, as are oppositely disposed rails 140. A nub 141 protrudes from this safety hook 138. The sensor 110 is enclosed in a housing 142, typically of two parts 142a, 142b. The lower part 142b is fitted with wings 144, that are configured correspondingly with respect to the rails 140, for engagement therewith, and includes an opening 146, for receiving the nub 141.

In an example operation, the sensor 110 is placed into the housing 142, and the housing 142 is slid in the direction of the arrow 147, such that the rails 140 engage the wings 144. Sliding continues until the nub 141 is received in the opening 146 with the sensor 110 over the safety hook 138. Once the adapter 26 is placed on the ultrasound transducer 23, or other imaging or scanning device, by adhesive affixation or the like, as shown in FIG. 8A, every insertion of the sensor 110 in the adapter 26 will bring the sensor 110 in the same position with respect to the ultrasound beam (or beam of the scanning or imaging device). Removal of the housing 142 from the adapter 26 is performed by pressing the tail 139 of the hook 138, and sliding the housing 142 out from the rails 140 and off of the base 130. The housing 142 can now be opened and the sensor 110 removed therefrom.

This design enables multiple insertion and extraction of the sensor in the medical device adapter in the exact or nearly exact position with respect to the ultrasound transducer 23. Therefore, calibration of the sensor to the ultrasound transducer 23 is necessary only once and thereafter (after saving the calibration parameters) there is no longer any need to repeat this calibration, unless the position of the adapter 26 has changed.

FIG. 8B shows the adapter 26 in use on a biopsy gun 150. This biopsy gun includes a needle 114 designed for rapid, typically spring loaded, advancement and retraction. Alternately, other devices may employ this adapter 26, as detailed for the above described apparatus.

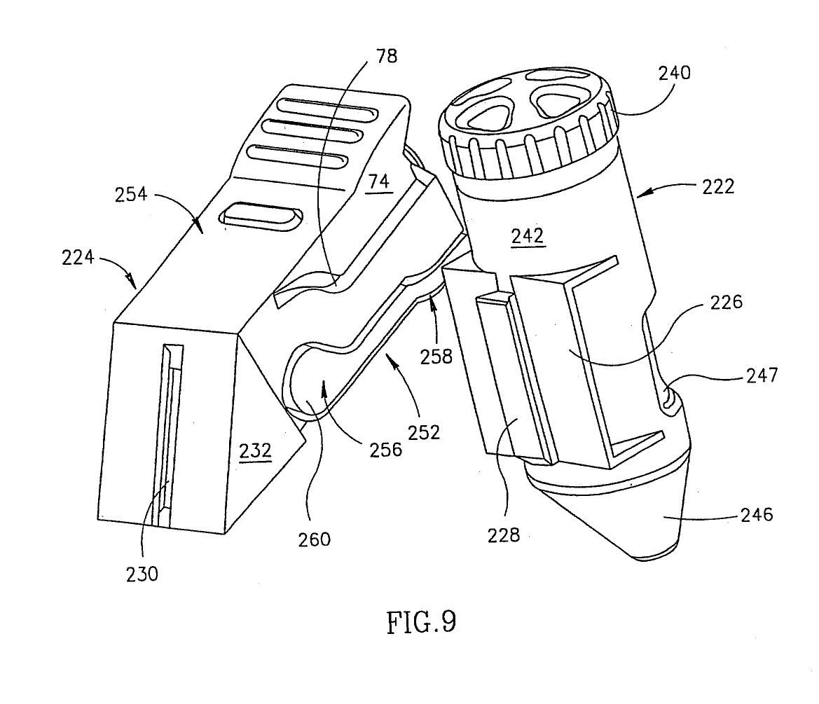

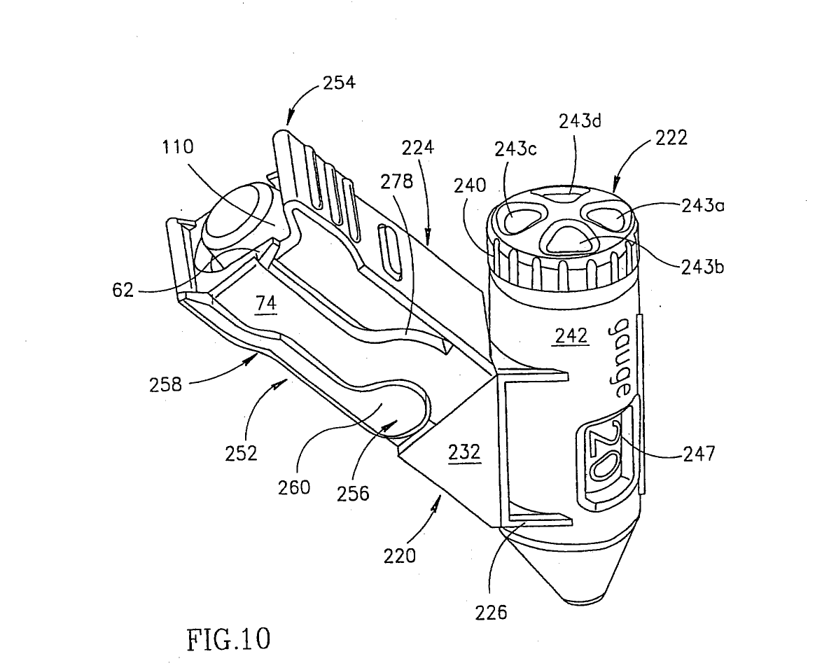

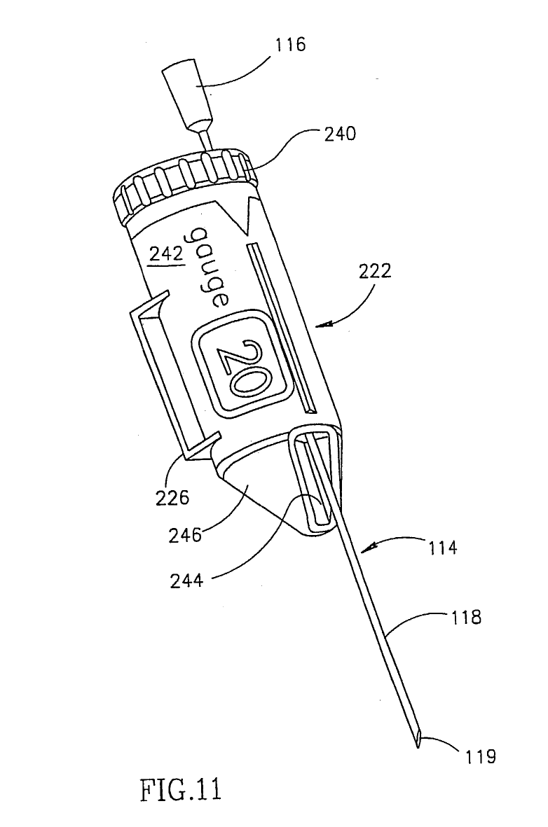

FIGS. 9-11 show a second embodiment of a needle adapter 220. This needle adapter 220 includes a needle housing 222 attached to a sensor housing 224. The attachment may be by a member 226 on the needle housing 222 having a protrusion 228 extending therefrom that is engaged by a slot 230 formed in member 232 attached to the sensor housing 224. Alternately, the positions of the protrusion 228 and slot 230 may be switched on the respective members 226, 232. It is preferred that this needle adapter be made of plastic or the like.

The needle housing 222 includes a rotatable cylinder 240, that fits within the body 242, preferably frictionally so as to permit the desired rotation. The cylinder 240 has bores 243a-243d (although fewer or more than four bores are also permissible), that can be configured to accommodate needles of various gauges. Upon rotation of the cylinder 240, each bore 243a-243d may be positioned so as to align with the bore 244 at the end 246 of the body, enabling the insertion of the needle 114 through the needle housing 222. The body 242 may also include a window 247, that for example, may display the gauge of the needle corresponding to the bore that has been rotated into position with the body bore 244.

The sensor housing 224 is similar to the corresponding portions of the needle adapter 20, detailed above, and is of similar construction and materials, except where indicated. This sensor housing 224 includes a main body 252 designed for attachment to a housing member 254 in a hinge-like manner. Operation of the sensor housing 224 is similar to the operation of the needle adapter 20 and alternatives (detailed above). The main body 252 includes a head 256 and a tail 258. The head 256 includes a cylinder member 260 for pivotal movement, when the sensor housing 224 is moved between open and closed positions (detailed above). The cylinder member 260 has a cylinder (not shown) intermediate platforms (not shown), similar to that detailed above and shown in FIGS. 2-6C. The cylinder of the cylinder member 260 accommodates a claw (not shown), similar to claw 70 (detailed above). The tail 258 is similar to the tail 50 of the main body portion 32, shown and detailed above, with structural elements labeled in accordance therewith.

The housing member 254 is similar to the housing member 34, shown and detailed above, with structural elements labeled in accordance therewith, except where noted. In particular, the lateral walls 74 are shaped to accommodate the cylinder member 260 of the main body portion 252. These walls 74 include a track 78, for accommodating the teeth 62 of the main body portion 252, when a locking arrangement for the sensor housing 224 is desired, similar to that detailed above.

Alternately, other sensor housings, such as a sensor housing similar to that used with the adapter 26 detailed above and shown in FIGS. 7A, 7B may be used in conjunction with the needle housing 222. In another alternate, the needle housing 222 could be replaced by a needle having a member similar to the member 226 with a protrusion thereon, for attaching to the sensor housing 224.

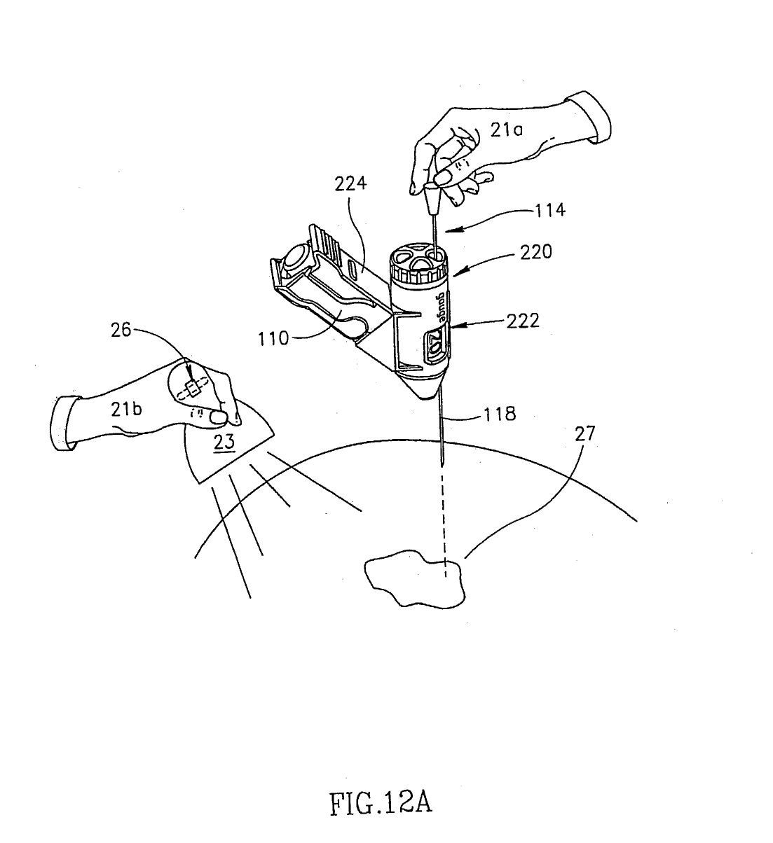

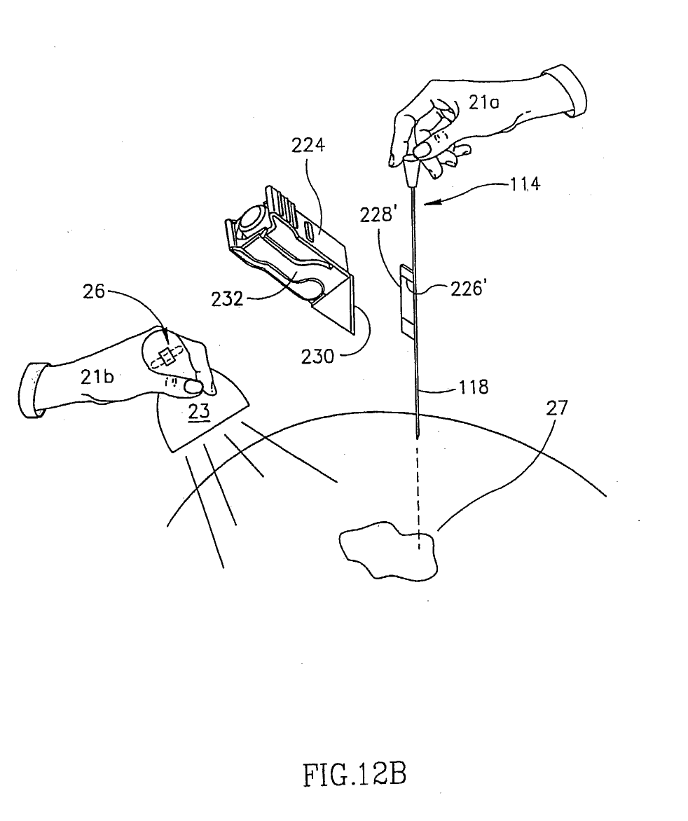

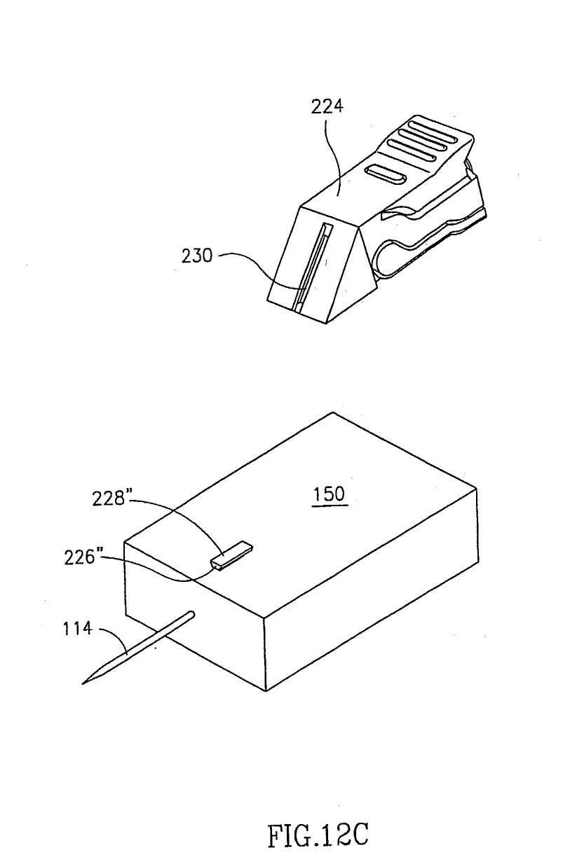

FIGS. 12A details an example operation of the needle adapter 220 in conjunction with an ultrasound transducer 23, employing the adapter 26 (as detailed above and shown in FIG. 8A above). The adapter 220 is placed on the body at a surgical site 27. The needle 114 is guided through the adapter 220. The exact length of the needle 114 that penetrates the body is not known, however, the orientation and the needle trajectory can be calculated with this adapter 220, since the orientation of the bore 243a-243d with respect to the position sensor 110 is known. An additional benefit from this adapter 220 is that the chance of bending the needle 114 is minimized during invasive operation, since a substantial length of the needle shaft 118 is contained in the respective bore 243a-243d.

FIG. 12B shows an alternate embodiment to the apparatus detailed in FIGS. 9-12A. This device is similar to that shown and described above for FIGS. 9-12A, with the needle adapter 220 replaced by a member 226′ having a protrusion 228′ (similar to protrusion 228) for engaging the slot 230 of member 232 of the sensor housing 224. As with the embodiment of FIGS. 9-12A, the structures on which the protrusion 228′ and slot 230 could also be switched.

FIG. 12C shows another embodiment, where the sensor housing 224 (detailed above in FIGS. 9-12A), attaches to a biopsy gun 150 (described above and detailed in FIG. 8B), for example, a 14G MAMMOTOME® probe with driver, as the biopsy gun 150 includes a member 226″ having a protrusion 228″ for engaging the slot 230 in the sensor housing 224. As with the embodiments of FIGS. 9-12B, the structures on which the protrusion 228″ and slot 230 rest, could also be switched.

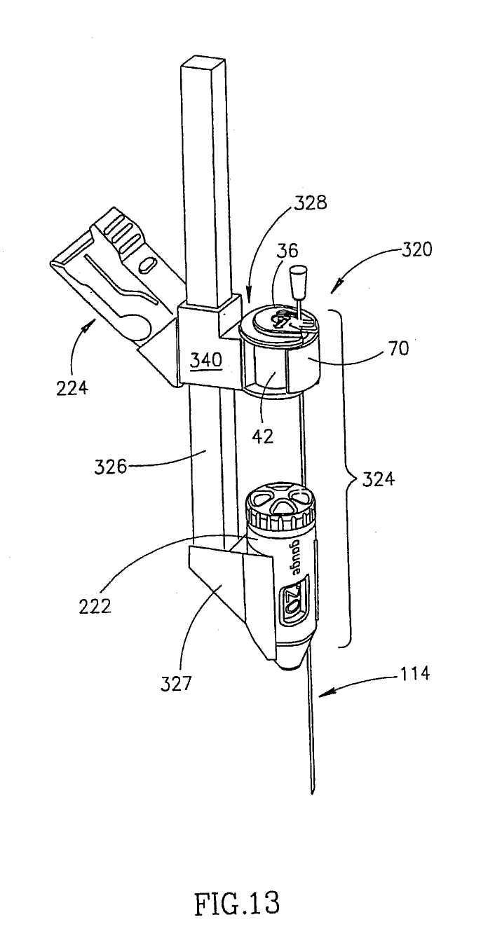

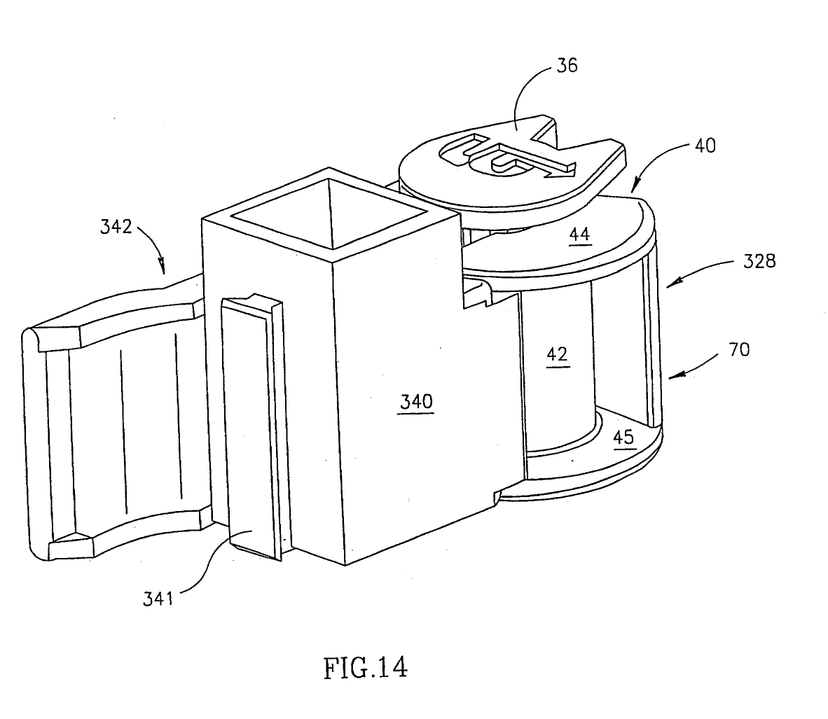

FIGS. 13 and 14 detail a needle guide 320. This needle guide 320 includes a needle alignment apparatus 324 and a sensor housing 224 (detailed above). The needle alignment apparatus 324 includes an arm 326, a needle housing 222 (detailed above) attached to an arm adapter 327, and a needle locker 328. The needle locker 328 includes components that are structurally similar to the those of the main body 32, the claw 70 of the housing member 34, and the pin 36, that have been detailed above. The needle locker 328 also includes an arm adapter 340 connected to the claw 70. This arm adapter 340 has a protrusion 341 thereon, for receipt in a slot (not shown) on the sensor housing 224. The locking mechanism for the needle 114 is similar to that detailed above for the first embodiment of the needle adapter (adapter 20). The tail 342, is similar to the tail 50 of the main body 32 (detailed above) and attaches to the arm adapter 340 by locking structures, such as a male-female engagement.

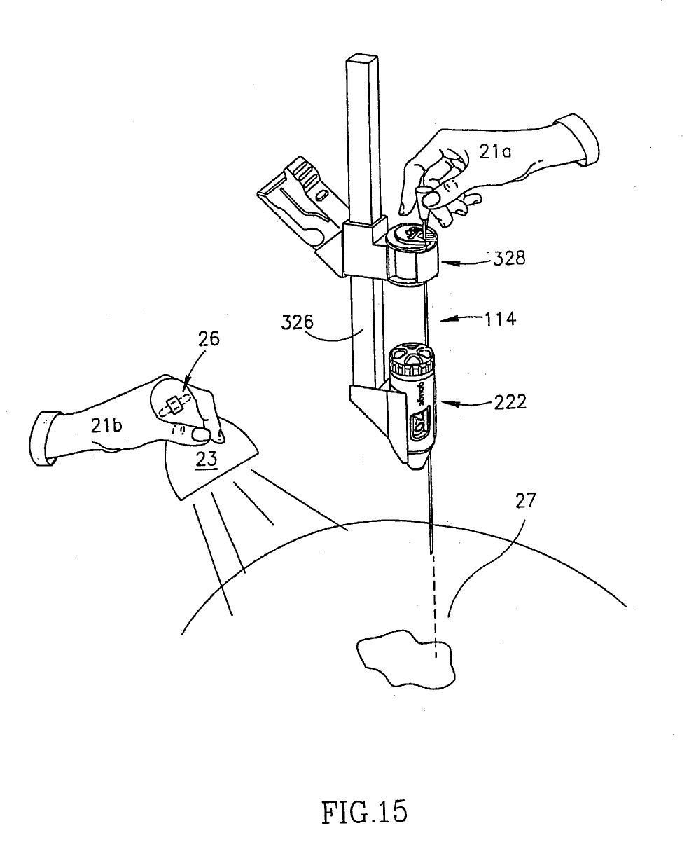

The locking mechanism of the needle 114 inside the needle locker 328 and the locking mechanism of the sensor housing 224 to the arm adapter 340 ensure that on each needle 114 insertion, the position of the needle 114 with respect to the sensor will be the same. This removes the need for calibrating the sensor to the needle 114 before performing a procedure. In addition, sliding the needle along the arm 326 minimizes the chance for needle bending. This effect is achieved since the needle is held on two solid devices co-aligned (the needle locker 328 and the needle holder 222, and because the needle holder is placed on the surgical site 27, as shown in FIG. 15.

In order to enable estimations of penetration depth of the needle 114, indicia may be added to the arm 326. These indicia are typically visible marks, and for example, may be in the form of a scale, such as inches, millimeters, etc.

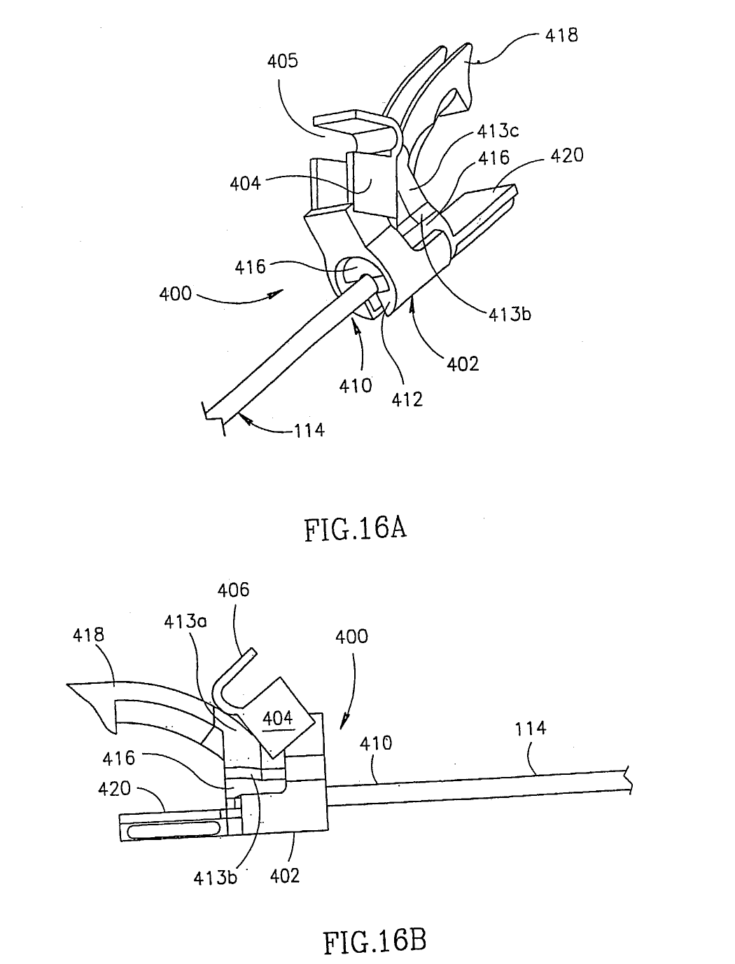

Reference is now made to FIGS. 16A and 16B. Here, there is an apparatus 400 adapted for receiving and engaging a MAMMOTOME® biopsy gun. The apparatus includes a needle housing 402, preferably adapted to engage a sensor housing 404. The needle housing 402 is sized and configured to receive a needle member 410. The sensor housing 404, includes a compartment 405, dimensioned to accommodate a sensor (detailed above), and includes a resilient flange 406 for keeping the sensor securely in the compartment 405.

This needle member 410 includes a needle (needle shaft) 114 and a needle extension 412 and member(s) 413a, 413b for attachment to the body of a MAMMOTOME® biopsy gun (not shown). The needle extension 412 comprises a shaft holder 416, an upper chamber wall 418 and a lower chamber wall 420.

The needle housing 402, and sensor housing 404, may be a single piece (one-piece construction) or removably attachable pieces (in accordance with that described above). The needle housing 402 is sized and configured (preferably with a cylindrical bore extending therethrough) to receive the needle extension 412, rather than directly attaching to the needle shaft 114. Particularly, the needle housing 402 is adapted for attaching to the shaft holder 416, while the shape of the sensor housing 404 is adapted to the shape of the upper chamber wall 418. All of these pieces (except for the needle 114) may be made of a resilient plastic or other material that has resiliency.

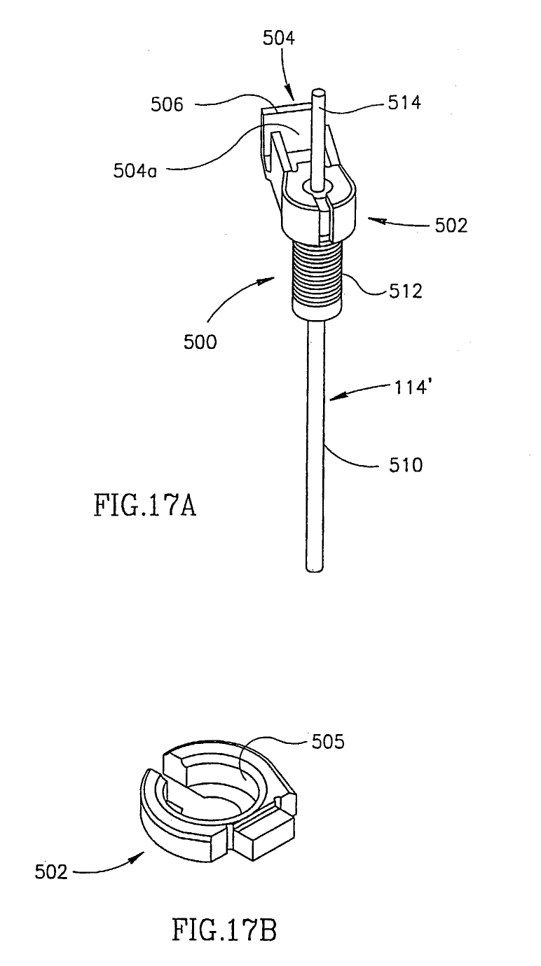

Reference is now made to FIGS. 17A and 17B. Here, there is an apparatus 500 adapted for receiving and engaging a SENOREX™ introducer 114′. The apparatus 500 includes a needle housing 502, preferably adapted to engage a sensor housing 504. The needle housing 502 is a C-shaped member with cut-out internal walls 505 adapted and dimensioned to engage and retain a collar portion of the SENOREX™ introducer 114′. The sensor housing 504, includes a compartment 504a, dimensioned to accommodate a sensor (detailed above), and includes a resilient flange 506 for keeping the sensor securely in the compartment 504a. The needle housing 502 is preferably configured to attach to the sensor housing 504, by any of the attachments detailed above, e.g., protrusion/slot or any other conventional attachments.

The introducer member 114′ includes an external shaft (not shown), a shaft portion 510, an extension 512 that serves as a shaft holder, and an upper portion 514, for connecting with the rest of the SENOREX™ aperture.

The needle housing 502, is sized and configured to receive the extension 512, rather than directly attaching to the shaft portion 510. The needle housing 502 and sensor housing 504, may be a single piece (one-piece construction). Similar to the apparatus 400 of FIGS. 16A and 16B (detailed above) the needle housing 502 and sensor housing may be made of a resilient plastic or other material that has resiliency.

While the invention has been described above, it is understood that the invention is not limited to the embodiments detailed above. The invention covers all modifications and equivalents within the spirit and scope thereof, as defined by the claims.

1. A needle adapter comprising:
a first member including a head portion and a tail portion, said head portion having a bore extending at least substantially therethrough and said head portion configured for receiving a segment of a second member and being configured for receiving a needle;
a second member having a segment and a tail portion, said segment attached to said tail portion and adapted for receipt in said head portion of said first member for attaching said first member and said second member in a hinge-like manner, allowing for movement of said first and second members between open and closed positions,
said tail portions of said first member and said second member being configured such that when said closed position is achieved, a volume for retaining a sensor is formed, and
a pin, removably attachable from said bore of said head portion of said first member, said pin for securing said needle upon its mounting in said needle adapter.

2. The needle adapter of claim 1, wherein said tail portions of each of said first and second members include cooperating locking structures thereon, such that said first member may be locked to the second member when in said closed position.

3. The needle adapter of claim 1, wherein said head portion of said first member includes a cylinder with a bore extending therethrough and said segment of said second member includes a claw for attaching to said cylinder.

4. The needle adapter of claim 3, wherein said cylinder is of a first radius of curvature and said claw is of a second radius of curvature, equal to or just slightly smaller than said first radius of curvature, such that said claw attaches to said cylinder by a frictional engagement, whereby said first member and said second member are moveable relative to each other about said cylinder.

5. A needle adapter comprising:
a first portion for holding a needle in communication with a second portion for housing a sensor, said second portion comprising:
a first member having a head portion and a tail portion, said head portion configured for receiving a segment of a second member;
a second member including a segment and a tail portion, said segment adapted for receipt by said head portion of said first member, such that said first member attaches to said second member in a hinge-like manner, allowing for movement of said first and second members between open and closed positions,
said tail portions of both said first member and said second member being configured such that when said closed position is achieved, a volume for retaining a sensor is formed.

6. The needle adapter of claim 5, wherein said tail portion of said first member and said second member include cooperating locking structures thereon, such that said first member may be locked to the second member when in said closed position.

7. The needle adapter of claim 5, wherein said head portion includes a cylinder member and said segment includes a claw.

8. A system for mounting a sensor comprising:
a base member having upper and lower surfaces;
a platform over said upper surface of said base member, said platform including oppositely disposed rails;
wings for attaching to oppositely disposed sides of a sensor housing, said wings being configured to be received and retained by said rails; and
a hook intermediate said rails on said platform for retaining said sensor housing, when said wings are retained in said rails.

9. The system of claim 8, wherein at least a portion of said lower surface includes adhesive.

10. A needle guide comprising:
a needle locker;
a needle holder below said needle locker and in alignment with said needle locker; and
a sensor housing in communication with said needle locker.

11. The needle guide of claim 10, wherein said sensor housing comprises:
a first member having a head portion and a tail portion, said head portion configured for receiving segment of a second member;
a second member including a segment and a tail portion, said segment adapted for receipt by said head portion of said first member, such that said first member attaches to said second member in a hinge-like manner, allowing for movement of said first and second members between open and closed positions,
said tail portions of both said first member and said second member being configured such that when said closed position is achieved, a volume for retaining a sensor is formed.

12. The needle adapter of claim 11, wherein said head portion includes a cylinder member and said segment includes a claw.

13. A system for positioning a sensor proximate a biopsy gun comprising:
a needle housing having a substantially cylindrical bore extending therethrough;
a sensor housing, said sensor housing removably attachable with respect to said needle housing;
a needle member adapted to be received in said housing, said needle member including a needle shaft in communication with a chamber, said chamber configured for accommodating a biopsy gun in communication with said needle shaft.

14. The system of claim 13, wherein said biopsy gun includes a MAMMOTOME® Biopsy gun.

15. An introducer receiving system comprising:
a collar member including cut-out inner walls, said collar member configured as a discontinuous ring and of a substantially resilient material so as to be configured for receiving an introducer member; and
a sensor housing, said sensor housing removably attachable with respect to said collar member.

16. The introducer of claim 15, wherein said collar member is configured for receiving a SENOREX™ introducer.
